(19) United States
(12) Patent Application Publication
(10) Pub. No.: US 2024/0130656 A1
(43) Pub. Date: Apr. 25, 2024

(54) BIOSIGNAL ELECTRODE

(71) Applicant: Murata Manufacturing Co., Ltd., Nagaokakyo-shi (JP)

(72) Inventors: Koji YOSHIDA, Smyrna, GA (US); Takeshi Torita, Nagaokakyo-shi (JP); Ichitaro Okamura, Nagaokakyo-shi (JP); Kosuke Sugiura, Nagaokakyo-shi (JP); Mika Fujiwaki, Nagaokakyo-shi (JP)

(21) Appl. No.: 18/480,702

(22) Filed: Oct. 3, 2023

Related U.S. Application Data
(60) Provisional application No. 63/417,392, filed on Oct. 19, 2022.

(51) Int. Cl. A61B 5/265; A61B 5/257; A61B 5/273; A61B 5/28; A61B 5/291

(52) U.S. Cl. CPC A61B 5/265; A61B 5/257; A61B 5/273; A61B 5/28; A61B 5/291; A61B 2560/04; A61B 2562/0215; A61B 2562/125

(57) ABSTRACT

A biosignal electrode that includes a substrate having first and second opposed main surfaces; a conductive material pattern on the first main surface of the substrate, the conductive material pattern defining a plurality of open spaces extending through the conductive material pattern and exposing the first surface of the substrate therethrough; and a biocompatible glue material within the plurality of open spaces of the conductive material pattern.

### CROSS REFERENCE TO RELATED APPLICATIONS

The present application claims priority to Provisional Patent Application No. 63/417,392, filed Oct. 19, 2022, the entire contents of which are incorporated herein by reference.

### FIELD OF THE INVENTION

The present invention relates to a biosignal electrode with a low impedance and an increased contact area for better signal transmission. More particularly, the present invention relates to a dry biosignal electrode with a low impedance and an increased contact area for better signal transmission.

### BACKGROUND OF THE INVENTION

Surface electrodes are types of electrodes applied to the skin of a subject for use in recording and evaluating the electrical activities of the heart (electrocardiography, i.e., ECG), skeletal muscles (electromyography, i.e., EMG) and neurons of the brain (electroencephalography, i.e., EEG) from the surface of the skin. The purpose of such a surface electrode is to act as a connector between a person's skin (where electrical signals are easiest to detect) and a detection unit via a lead cable.

There are two general types of surface electrodes, dry and wet. Dry electrodes are a type of surface electrode that do not use an aqueous electrolyte. One type of dry electrode has a simple structure that includes a metal plate connected with a lead wire. Other types of dry electrodes have a silicone elastomer base material with a conductive powder contained therein, and an electrode connector on the silicone elastomer for connection to the lead cable. These dry electrodes also typically include a conductive adhesive gel on one side for adhering to the skin. The conductive adhesive gel presents unique problems of having some patients being allergic to the gel electrolyte, that the conductive adhesive gel has a relatively short shelf-life due to drying out of the gel, and the use of a dried out conductive adhesive gel can also cause skin irritation to the person's skin.

If a dry electrode makes good contact with the skin, the signals that are generated will be relatively accurate. Thus, one of the most important goals for any such dry electrode is to obtain a low contact impedance in order to attain a high signal-to-noise ratio. However, if an electrode does not adhere well to the skin or does not have a sufficient contact area with the skin, the resulting signal may contain an undue amount of noise which will interfere with accurate measurement.

Due to the deficiencies in present dry electrode designs, one known way to improve the contact impedance is to abrade the skin of the person so as to remove a layer of dead skin from the surface. This technique, however, can cause irritation to the skin of the person and is therefore not desirable.

Wet electrodes are typically constructed to have one or more pins that extend outward from the bottom of the electrode. These electrodes are then attached to the skin of a person with an elastic band after an aqueous electrode gel is placed between the electrode and the skin of the person. Such a system is uses additional materials and steps, can be messy with the use of the aqueous electrode gel, and more importantly, typically causes irritation to the skin of the person by the pins pressing into the skin.

### SUMMARY OF THE INVENTION

Thus, an object of the present invention is to provide a biosignal electrode with a low impedance and an increased contact area for better signal transmission, and more particularly a dry biosignal electrode that has a low impedance and that does not utilize a conductive adhesive gel in order to have a low signal-to-noise ratio.

This summary is provided to introduce a selection of concepts in a simplified form that are further described below in the DETAILED DESCRIPTION. This summary is not intended to identify key features of the claimed subject matter, nor is it intended to be used as an aid in determining the scope of the claimed subject matter.

According to an aspect of the present invention, the biosignal electrode includes a substrate having first and second opposed main surfaces; a conductive material pattern on the first main surface of the substrate, the conductive material pattern defining a plurality of open spaces extending through the conductive material pattern and exposing the first surface of the substrate therethrough; and a biocompatible glue material within the plurality of open spaces of the conductive material pattern.

In a further preferred embodiment, the conductive material is MXene and the substrate is a thermoplastic polyurethane material.

In another aspect of the present invention, the conductive material pattern has a larger thickness than the biocompatible glue.

Additional advantages and novel features of the present invention will be set forth in part in the description that follows, and in part will become more apparent to those skilled in the art upon examination of the following or upon learning by practice of the disclosure.

### BRIEF DESCRIPTION OF THE DRAWINGS

The novel features believed to be characteristic of the disclosure are set forth in the appended claims. In the description that follows, like parts are marked throughout the specification and drawings with the same numerals, respectively. The drawing figures are not necessarily drawn to scale and certain figures may be shown in exaggerated or generalized form in the interest of clarity and conciseness. The disclosure itself, however, as well as a preferred mode of use, further objects and advances thereof, will be best understood by reference to the following detailed description of illustrative aspects of the disclosure when read in conjunction with the accompanying drawings, wherein:

FIG. 1 is a bottom plan view showing a biosignal electrode of a first embodiment of the present invention;

FIG. 2 is an enlarged view of area A in FIG. 1;

FIG. 3 is a cross section through line B-B of FIG. 2;

FIGS. 4-9 show various alternate shapes of the conductive material pattern;

FIG. 10 is a bottom plan view showing a biosignal electrode of a second embodiment;

FIG. 11 is an enlarged view of area C in FIG. 10;

FIG. 12 is a cross section through line D-D of FIG. 10;

FIG. 13 is a bottom plan view showing a portion of a biosignal electrode of a third embodiment; and

FIG. 14 is a cross section along line D-D of FIG. 13.

### DETAILED DESCRIPTION OF THE PREFERRED EMBODIMENTS

The detailed description set forth below in connection with the appended drawings is intended as a description of various configurations and is not intended to represent the only configurations in which the concepts described herein may be practiced. The detailed description includes specific details for the purpose of providing a thorough understanding of such various concepts. However, it will be apparent to those skilled in the art that these concepts may be practiced without these specific details. In some instances, well known structures and components are shown in block diagram form in order to avoid obscuring such concepts. Each embodiment is exemplified below, and it will be apparent that the structures shown in different embodiments can be partly replaced or combined with each other. In the second and subsequent embodiments, description of matters common to the first embodiment will be eliminated, and only different points will be described. In particular, a similar effect by a similar structure will not be sequentially referred to for each embodiment.

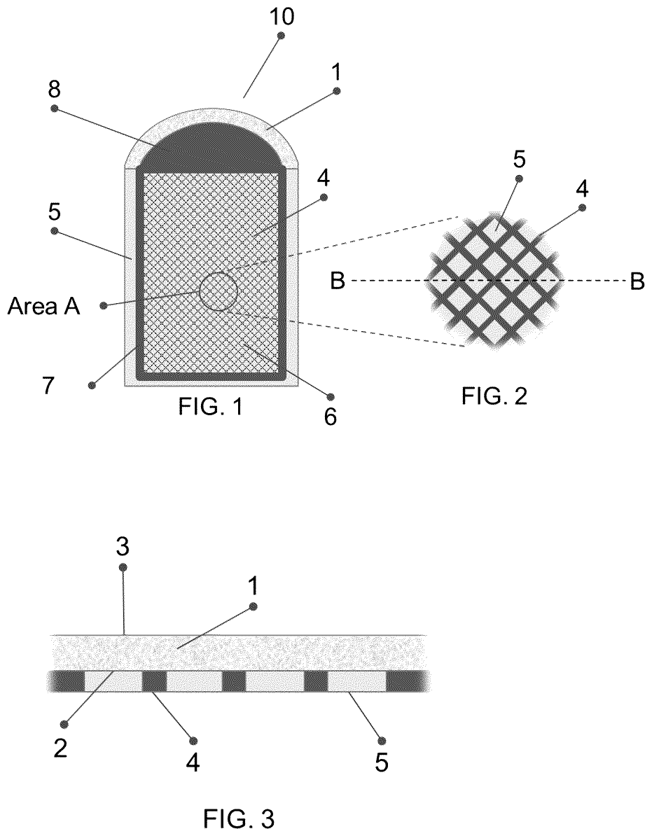

Referring now to FIGS. 1-3, a first embodiment of the present invention will be described. FIG. 1 is a bottom plan view showing a biosignal electrode of a first embodiment, FIG. 2 is an enlarged view of area A in FIG. 1; and FIG. 3 is a cross section through line B-B of FIG. 2.

In the embodiment of FIGS. 1-3, the substrate is configured such that the biosignal electrode 10 is a tab-type electrode. As shown in FIGS. 1-3, the biosignal electrode 10 includes a substrate 1 having first and second opposed main surfaces 2, 3, a conductive material pattern 4 on the first main surface 2 of the substrate 1, and a biocompatible glue material 5. The preferred material for the substrate 1 is a thermoplastic polyurethane. The substrate can also be made of other materials, such as, for example, PET, PEN, polyimide, LCP, PAN, PMMA, PVDF, POE, PTFE, PE, PP, nylon, paper, nonwoven fabrics, PDMS, or silicone. Preferably, the substrate 1 has a thickness of 0.1 mm to 2.0 mm. The thickness of the substrate, however, is not critical and one skilled in the art will appreciate that the thickness of the substrate will depend on the nature of use of the biosignal electrode. For example, when used for skin contact, the thickness will be set such that the biosignal electrode has sufficient flexibility to follow the contours of the surface of the skin.

As shown in FIGS. 1-3, the conductive material pattern 4 on the first main surface 2 of the substrate 1 defines a plurality of open spaces that extend through the conductive material pattern 4 to expose the first main surface 2 of the substrate 1 therethrough. In the embodiment of FIGS. 1-3, the conductive material pattern has a mesh portion 6, a frame portion 7 surrounding the mesh portion, and a lead-out electrode portion 8. The mesh portion 6 preferably has a line width of 0.01 to 5 mm, and most preferably 0.1 to 1 mm. Preferably, an area occupied by the mesh portion 6 is more than 50% of an entire area of the first main surface 2 of the substrate 1. Further, and although not shown in the drawings, the frame portion 7 can be omitted and the mesh portion 6 can be provided to extend to outer edges of the substrate 1.

Preferably, the conductive material pattern 4 has a thickness of 0.1 mm to 2.0 mm. The thickness of the conductive material pattern 4, however, is not critical and one skilled in the art will appreciate that the thickness of the conductive material pattern will depend on the nature of use of the biosignal electrode. For example, when used for skin contact, the thickness will be set such that the biosignal electrode has sufficient flexibility to follow the contours of the surface of the skin.

The conductive material of the conductive material pattern 4 preferably comprises at least one of Ag, Au, Cu, Al, Be, Mg, Ca, Na, Rh, Ir, carbon, carbon nanotubes, and graphene. While the particle size of the conductive material is not particularly limited, the conductive material preferably has an average particle size set such that the conductive material can be adequately formed on the substrate so that electrical conductivity is maintained. Preferably, the conductive material has a D50 cumulative particle size distribution from 100 μm to 100 nm. Depending on the material of the conductive material, the particles do not necessarily have to physically touch each other within the conductive material pattern. All that is required is that the material, size, and arrangement of the conductive material pattern on the substrate be such that an electrical signal can be propagated to the lead-out electrode portion 8 from the mesh portion 6 and/or the frame portion 7.

With this in mind, a most preferred material for the conductive material is MXene. MXene is particularly preferred because it is a two-dimensional material having a low dielectric constant, and can be easily formed on the surface of the substrate. MXene is also particularly preferred because its two-dimensional arrangement can maintain conductivity even when the substrate is stretched or bent. This is particularly useful in skin electrodes that need to conform to the different topologies of the skin of different people. The MXene particles preferably have a D50 cumulative particle size distribution of 20 μm to 500 μm, more preferably from 30 μm to 300 μm, as measured with an LA960 Laser Scattering Particle Size Distribution Analyzer available from Horiba, Ltd.

The conductive material pattern 4 can be placed on the surface of the substrate by various methods, including, but not limited to, creating a paste with the conductive material included therein and then using screen printing, inkjet printing, flexographic printing, gravure printing, or the like, to form the conductive material pattern on the substrate. The particular method selected will depend on the conductive material pattern shape and width and thickness of the lines. For example, for lines having a width of more than 1 mm, a screen printing method can be used where the squeeze angle is 60°, the ink application speed is 150 mm/s at a print head distance of 1.5 mm when using a conductive paste containing 30-35 wt % of a Ag solid conductive material, and having a viscosity of 350-400 Pa-s and a density of 1.6 g/cm³.

The lead-out electrode portion 8 is provided for connection to a lead cable (not shown) which is then connected to a detection unit. The use of a lead-out electrode portion is optional and the lead cable can be directly secured to the mesh portion 6 and/or the frame portion 7. When a lead-out electrode portion 8 is provided, it is preferred that the lead-out electrode portion 8 be made from the same material as the mesh portion 6, and thus also formed by a printing process such as screen printing, flexographic printing, gravure printing, and the like.

As shown in FIG. 1, the biocompatible glue material 5 is located within the plurality of open spaces of the mesh portion 6, and is also preferably located between the frame portion 7 and an outer edge of the substrate 1. The biocompatible glue material is preferably selected from a hot melt glue (such as a styrene-isoprene-styrene-block copolymer), an acrylic-based glue, a UV curable glue (such as a bismaleimide glue), a synthetic rubber-based glue, a silicone-based glue, and a urethane-based glue. Similar to the conductive material pattern 4, the biocompatible glue material 5 can be placed within the plurality of open spaces of the mesh portion 6 and/or placed between the frame portion 7 and the outer edges of the substrate 1 by various methods, including, but not limited to, screen printing, inkjet printing, flexographic printing, gravure printing, or the like. The biocompatible glue material preferably has a thickness that is the same as or less than that of the conductive material pattern 4. Most preferably, the thickness of the biocompatible glue material 5 is less than that of the conductive material pattern 4 so as to ensure good contact of the conductive material pattern with the surface of the skin.

With the structure described above with reference to FIGS. 1 and 2, a biosignal electrode with a low impedance and an increased contact area for better signal transmission can be provided.

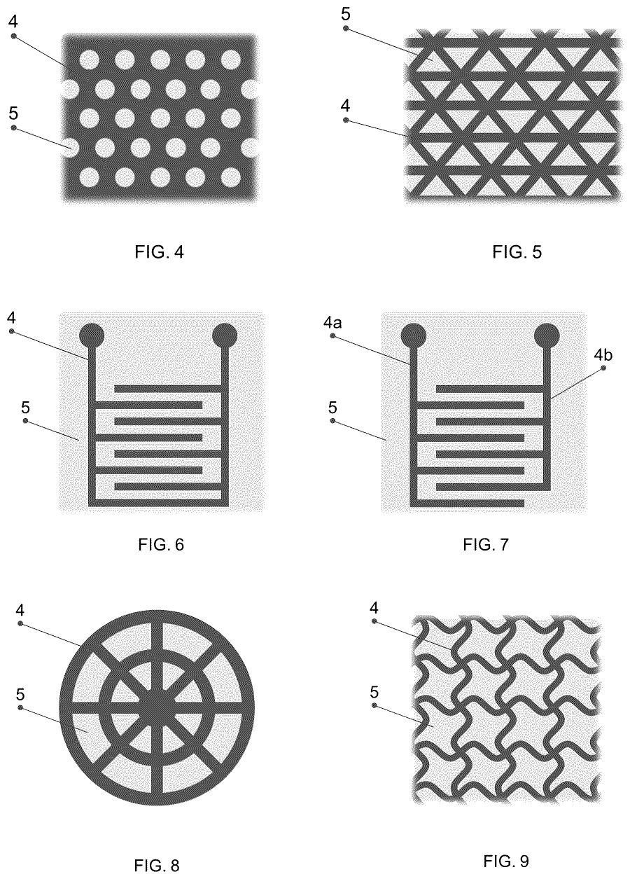

Referring now to FIGS. 4-9, modifications of the shape of the conductive material pattern are shown. These modifications can include the conductive material pattern 4 being shaped such that the plurality of openings for the biocompatible glue material 5 are round (FIG. 4); the conductive material pattern 4 being shaped such that the plurality of openings for the biocompatible glue material 5 are triangular (FIG. 5); the conductive material pattern 4 being shaped in the form of a unitary inter-digital electrode pattern where the biocompatible glue material 5 surrounds and/or is between the lines of the inter-digital electrode pattern (FIG. 6); the conductive material pattern 4 being shaped in the form of a divided electrode inter-digital electrode pattern where the biocompatible glue material 5 surrounds and/or is between the lines of the inter-digital electrode pattern (FIG. 7); the conductive material pattern 4 being shaped in a circular pattern having a plurality of concentric rings and spokes, and where the biocompatible glue material 5 is present within the openings defined by the concentric rings and spokes (FIG. 8); and the mesh portion of conductive material pattern 4 has a plurality of wave-shaped electrode lines that define the plurality of openings for the biocompatible glue material 5 (FIG. 9). These are merely alternative examples of possible patterns for the conductive material pattern 4.

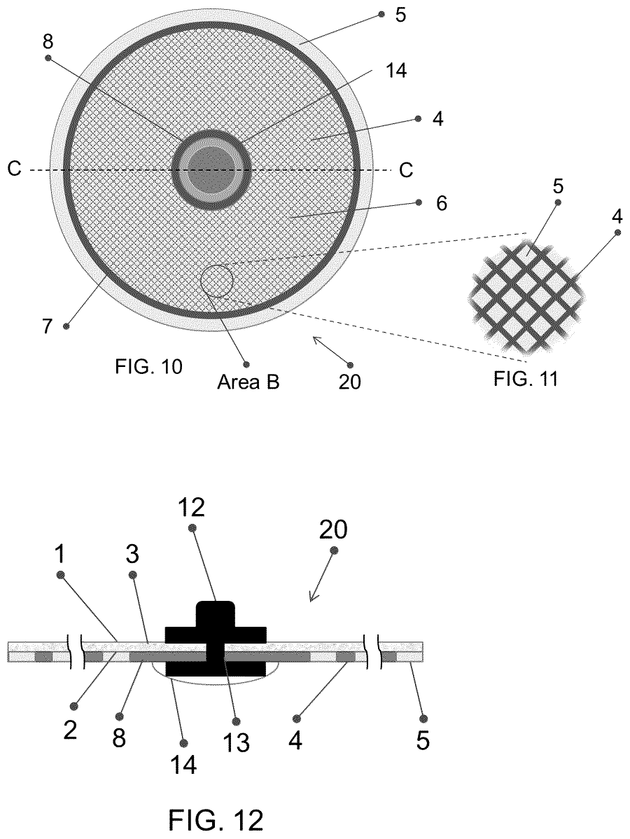

Referring now to FIGS. 10-12, a second embodiment of the present invention will now be described. FIG. 10 is a bottom plan view showing a biosignal electrode 20 of a second embodiment, FIG. 11 is an enlarged view of area C in FIG. 10; and FIG. 12 is a cross section through line D-D of FIG. 10.

In the embodiment of FIGS. 10-12, the substrate 1 is configured such that the biosignal electrode 20 is a snap-type electrode. As shown in FIGS. 10-12, and similar to the first embodiment of FIGS. 1-3, the biosignal electrode 20 includes a substrate 1 having first and second opposed main surfaces 2, 3, a conductive material pattern 4 on the first main surface 2 of the substrate 1, and a biocompatible glue material 5. Details of the materials, dimensions, etc., for these portions of the biosignal electrode 20 are omitted from the description of the second embodiment as they are the same as those of the first embodiment, and like reference numerals are used for like features in the figures.

Different form the first embodiment, the biosignal electrode 20 includes a centrally-located lead-out electrode 12. The lead-out electrode 12 is preferably composed of a conductive carbon plastic material and extends through a via hole 13 in a central portion of the substrate 1 and the conductive material pattern 4. The lead-out electrode 12 is provided for connection to a lead cable (not shown) which is then connected to a detection unit. Preferably, and for ease of assembly, the lead-out electrode 12 is formed in two separate pieces (not explicitly shown in FIG. 12) that are fit together through the through-hole 13. Additionally, a cover 14 preferably extends across the exposed surface of the lead-out electrode 12 on the side of the conductive material pattern 4 so that the cover 14 is located between the conductive carbon plastic material of the lead-out electrode 12 and the skin of the wearer. The use of such a cover prevents the creation of an unstable potential between the conductive carbon plastic of the lead-out electrode and the skin of the wearer. In place of the cover 14, an Ag/AgCl coated conductive carbon plastic can be used, and also provides a stable potential.

Similar to the first embodiment, the conductive material pattern 4 of the second embodiment of FIGS. 10-12 defines a plurality of open spaces that extend through the conductive material pattern 4 to expose the first main surface 2 of the substrate 1 therethrough. In the embodiment of FIGS. 10-12, the conductive material pattern also has a mesh portion 6, a frame portion 7 surrounding the mesh portion, and a lead-out electrode portion 8 that provides conductivity to the lead-out electrode 12. The biocompatible glue material 5 is located within the plurality of open spaces of the mesh portion 6, and is also preferably located between the frame portion 7 and an outer edge of the substrate 1.

It should also be noted that the shape of the conductive material pattern shown in FIG. 10 can be modified and shaped in accordance with the patterns shown in FIGS. 4-9.

With the structure described above with reference to FIGS. 10-12, a biosignal electrode with a low impedance and an increased contact area for better signal transmission can be provided.

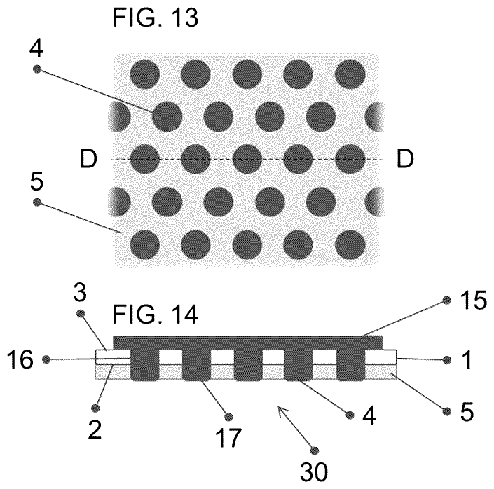

Referring now to FIGS. 13 and 14, a third embodiment of the present invention will be described. FIG. 13 is a bottom plan view showing a portion of the biosignal electrode of the third embodiment, and FIG. 14 is a cross section along line D-D of FIG. 13, and showing a cross section of the biosignal electrode.

As shown in FIGS. 13 and 14, and similar to the first embodiment of FIGS. 1-3, the biosignal electrode 30 includes a substrate 1 having first and second opposed main surfaces 2, 3, a conductive material pattern 4 on the first main surface 2 of the substrate 1, and a biocompatible glue material 5. In the biosignal electrode 30 of the third embodiment, the substrate 1 includes a plurality of through holes 16 that extend between the first and second opposed main surfaces 2, 3 of the substrate 1. The through holes 16 preferably each have a diameter of 0.5 to 5.0 mm, and most preferably more than 1 mm. The through holes 16 are filled with a conductive material 17 that extends between the first and second opposed main surfaces 2, 3, and beyond the first main surface 2 to define a conductive material pattern 4 at the first main surface of the substrate. The conductive material 17 provides an electrically conductive path between the first and second opposed main surfaces 2, 3. Preferably, and as shown in FIG. 13, the conductive material pattern 4 is in the form of a plurality of round shaped electrode portions. Preferably, an area occupied by the plurality of round shaped electrode portions is equal to or more than 50% of an entire area of the first or the second main surfaces of the substrate.

The lead-out electrode 15 on the second main surface 3 of the substrate 1 electrically connects to the conductive material 17 within the each of the plurality of through holes 16. The lead-out electrode 18 is provided for connection to a lead cable (not shown) which is then connected to a detection unit.

Details of the materials, dimensions, etc., for these portions of the biosignal electrode 30 are omitted from the description of the third embodiment as they are the same as those of the first and second embodiments, and like reference numerals are used for like features in the figures.

With the structure described above with reference to FIGS. 13 and 142, a biosignal electrode with a low impedance and an increased contact area for better signal transmission can be provided.

While the invention herein has been described in connection with a biosignal electrode for biosignal measurement, the present invention is also equally useable in other types of sensors such as, for example, gas sensors, ion sensors, etc. Thus, while the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

What is claimed is:

1. A biosignal electrode comprising:
a substrate having first and second opposed main surfaces;
a conductive material pattern on the first main surface of the substrate, the conductive material pattern defining a plurality of open spaces extending through the conductive material pattern and exposing the first surface of the substrate therethrough; and
a biocompatible glue material within the plurality of open spaces of the conductive material pattern.

2. The biosignal electrode of claim 1, wherein the conductive material is MXene.

3. The biosignal electrode of claim 2, wherein the MXene has a D50 cumulative particle size distribution of 20 μm to 500 μm.

4. The biosignal electrode of claim 1, wherein the conductive material comprises at least one of Ag, Au, Cu, Al, Be, Mg, Ca, Na, Rh, Ir, carbon, carbon nanotubes, and graphene.

5. The biosignal electrode of claim 1, wherein the substrate comprises thermoplastic polyurethane, PET, PEN, polyimide, LCP, PAN, PMMA, PVDF, POE, PTFE, PE, PP, nylon, paper, nonwoven fabrics, PDMS, or silicone.

6. The biosignal electrode of claim 1, wherein the conductive material pattern has a larger thickness than the biocompatible glue.

7. The biosignal electrode of claim 1, further comprising a lead-out electrode portion on the first main surface of the substrate and electrically connected to the conductive material pattern.

8. The biosignal electrode of claim 1, wherein the substrate is in a tab shape.

9. The biosignal electrode of claim 1, wherein the substrate is circular.

10. The biosignal electrode of claim 1, wherein the conductive material pattern is in a mesh shape.

11. The biosignal electrode of claim 1, wherein the plurality of open spaces are triangular.

12. The biosignal electrode of claim 1, wherein the plurality of open spaces are circular.

13. The biosignal electrode of claim 1, wherein the conductive material pattern is in an inter-digital shape.

14. The biosignal electrode of claim 1, wherein the conductive material pattern is in a circular shape.

15. The biosignal electrode of claim 14, wherein the circular shape includes a mesh shape portion.

16. The biosignal electrode of claim 14, wherein the circular shape includes a plurality of concentric ring patterns.

17. The biosignal electrode of claim 1, wherein the conductive material pattern includes a plurality of wave-shaped electrode lines.

18. The biosignal electrode of claim 1, wherein the biocompatible glue is made of a material selected from a hot melt glue, an acrylic-based glue, a UV curable glue, a synthetic rubber-based glue, a silicone-based glue, and a urethane-based glue.

19. The biosignal electrode of claim 1, wherein the substrate includes a via hole in a central portion of the substrate, and the biosignal electrode further comprises a centrally-located lead-out electrode that extends through the via hole and is electrically connected to the conductive material pattern.

20. The biosignal electrode of claim 19, wherein the lead-out electrode is composed of a conductive carbon plastic material.

21. A biosignal electrode comprising:
a substrate having first and second opposed main surfaces and a plurality of through holes extending from the first main surface to the second main surface;
a conductive material within the through holes and extending from the second main surface to the first main surface and extending beyond the first surface to define a conductive material pattern at the first main surface of the substrate, the conductive material pattern defining a plurality of open spaces extending through the conductive material pattern and exposing the first surface of the substrate therethrough; and
a biocompatible glue material within the plurality of open spaces of the conductive material pattern at the first surface of the substrate.

22. The biosignal electrode of claim 21, wherein the conductive material pattern includes a plurality of round shaped electrode portions.

23. The biosignal electrode of claim 21, wherein the conductive material is MXene.

24. The biosignal electrode of claim 23, wherein the MXene has a D50 cumulative particle size distribution of 20 μm to 500 μm.

25. The biosignal electrode of claim 21, wherein the conductive material comprises at least one of Ag, Au, Cu, Al, Be, Mg, Ca, Na, Rh, Ir, carbon, carbon nanotubes, and graphene.

26. The biosignal electrode of claim 21, wherein the substrate comprises thermoplastic polyurethane, PET, PEN, polyimide, LCP, PAN, PMMA, PVDF, POE, PTFE, PE, PP, nylon, paper, nonwoven fabrics, PDMS, or silicone.

27. The biosignal electrode of claim 21, wherein the conductive material pattern has a larger thickness than the biocompatible glue.

28. The biosignal electrode of claim 21, further comprising a lead-out electrode portion on the second surface of the substrate and connecting the electrode material within the plurality of through holes at the second surface of the substrate.

29. The biosignal electrode of claim 21, wherein the biocompatible glue is made of a material selected from a hot melt glue, an acrylic-based glue, a UV curable glue, a synthetic rubber-based glue, a silicone-based glue, and a urethane-based glue.
